(19) United States
(12) Patent Application Publication
(10) Pub. No.: US 2007/0173840 A1
(43) Pub. Date: Jul. 26, 2007

(54) Bone plate with cover

(76) Inventor: Randall J. Huebner, Beaverton, OR (US)

Correspondence Address: KOLISCH HARTWELL, P.C., 200 PACIFIC BUILDING, 520 SW YAMHILL STREET, PORTLAND, OR, 97204, US

(21) Appl. No.: 11/330,802

(22) Filed: Jan. 11, 2006

(51) Int. Cl. A61F 2/30

(52) U.S. Cl. 606/69

(57) ABSTRACT

System, including methods, apparatus, and kits, for fixing bones with a bone plate and a cover plate attached over the bone plate.

### CROSS-REFERENCES TO RELATED APPLICATIONS

This application incorporates herein by reference the following U.S. patent applications: Ser. No. 10/716,719, filed Nov. 19, 2003; Ser. No. 10/717,015, filed Nov. 19, 2003; Ser. No. 10/717,399, filed Nov. 19, 2003; Ser. No. 10/717,401, filed Nov. 19, 2003; Ser. No. 10/717,402, filed Nov. 19, 2003; Ser. No. 10/731,173, filed Dec. 8, 2003; Ser. No. 10/873,522, filed Jun. 21, 2004; Ser. No. 10/968,850, filed Oct. 18, 2004; Ser. No. 11/071,050, filed Feb. 28, 2005; and Ser. No. 11/109,985, filed Apr. 19, 2005.

### INTRODUCTION

The human skeleton is composed of 206 individual bones that perform a variety of important functions, including support, movement, protection, storage of minerals, and formation of blood cells. To ensure that the skeleton retains its ability to perform these functions, and to reduce pain and disfigurement, bones that become damaged should be repaired promptly and properly. Typically, a fractured or cut bone is treated using a fixation device, which reinforces the bone and keeps it aligned during healing. Fixation devices may include external fixation devices (such as casts and/or fixators) and/or internal fixation devices (such as bone plates, nails, and/or bone screws), among others.

Bone plates are sturdy internal devices, often made of metal, that mount directly to the bone, under the skin and other soft tissue, adjacent a fracture (or other bone discontinuity). To use a bone plate to repair a bone discontinuity, a surgeon typically (1) selects an appropriate plate, (2) reduces the discontinuity (e.g., sets the fracture), and (3) fastens the plate to bone fragments disposed on opposite sides of the discontinuity using suitable fasteners, such as screws and/or wires, so that the bone plate spans the discontinuity and the bone fragments are fixed in position.

Bone plates are generally installed in contact with bone and thus under soft tissue, such as tendon and muscle. Since the bone plates may be fixed in relation to bone, overlying soft tissue should be able to slide freely over the outer surface of the bone plate to permit unimpaired body movement. However, soft tissue over bone plates may be irritated and/or injured by repetitively sliding over the plates.

### SUMMARY

The present teachings provide a system, including methods, apparatus, and kits, for fixing bones with a bone plate and a cover plate attached over the bone plate.

### BRIEF DESCRIPTION OF THE DRAWINGS

FIG. 1 is an exploded view of an exemplary system for fixing bones with a bone plate and a cover plate that attaches to the bone plate, in accordance with aspects of the present teachings.

FIG. 2 is a sectional view of another exemplary bone plate and another exemplary cover plate secured over the bone plate using a threaded fastener, with the cover plate extending over an outer edge of the bone plate, in accordance with aspects of the present teachings.

FIG. 3 is a sectional view of yet another exemplary bone plate and yet another exemplary cover plate secured over the bone plate using a threaded fastener, with the cover plate including an internal flange that extends into the bone plate, in accordance with aspects of the present teachings.

FIG. 4 is a sectional view of another exemplary bone plate and another exemplary cover plate secured over the bone plate using a snap-fit mechanism, in accordance with aspects of the present teachings.

FIG. 5 is a volar view of a distal region of a fractured radius bone with an exemplary bone plate secured to the radius bone without a cover plate, in accordance with aspects of the present teachings.

FIG. 6 is a volar view of the radius bone and bone plate of FIG. 5 with an exemplary cover plate secured over the bone plate, in accordance with aspects of the present teachings.

FIG. 7 is a sectional view of the radius bone, bone plate, and cover plate of FIG. 6, taken generally along line 7-7 of FIG. 6.

FIG. 8 is a fragmentary view of selected portions of FIG. 7, taken generally at “8” in FIG. 7, with a tendon extending over the bone plate and cover plate, in accordance with aspects of the present teachings.

FIG. 9 is a fragmentary view of the bone plate and cover plate of FIG. 6, taken from the opposing direction and toward an inner surface of the bone plate in the absence of bone and fasteners, in accordance with aspects of the present teachings.

### DETAILED DESCRIPTION

The present teachings provide a system, including methods, apparatus, and kits, for fixing bones with a bone plate and a cover plate attached over the bone plate. The bone plate may be configured for use on any suitable bone and bone region. In some embodiments, the bone plate may be structured for use near an end of a bone, where soft tissue irritation may be a greater problem, such as a distal end region of a radius bone. The cover plate may be secured to the bone plate with at least one threaded fastener, with only one threaded fastener, or with no threaded fastener. The cover plate may cover any suitable portion of the bone plate, such as some, most, or at least substantially all of a head portion of the bone plate. In some embodiments, the head portion may be covered selectively by the cover plate relative to other portions of the bone plate. The cover plate may include a flange that extends transversely from an inner surface of the body of the cover plate, generally toward the bone plate and bone, to restrict pivotal motion of the cover plate. The flange may extend into the bone plate, for example, a pin that extends into an opening of the bone plate, and/or may extend over an edge of the bone plate, such as a lip of the cover plate that extends along the perimeter of the bone plate. In any case, the cover plate of the present teachings may reduce abrasion of overlying soft tissue relative to a bone plate alone and/or relative to other cover plates, by offering a smoother and/or more continuous surface for contact with the soft tissue.

Further aspects of the present teachings are described in the following sections, including (I) overview of exemplary fixation systems, (II) bone plates, (III) cover plates, (IV) kits for bone fixation, (V) application of fixation systems, and (VI) example.

### I. OVERVIEW OF EXEMPLARY FIXATION SYSTEMS

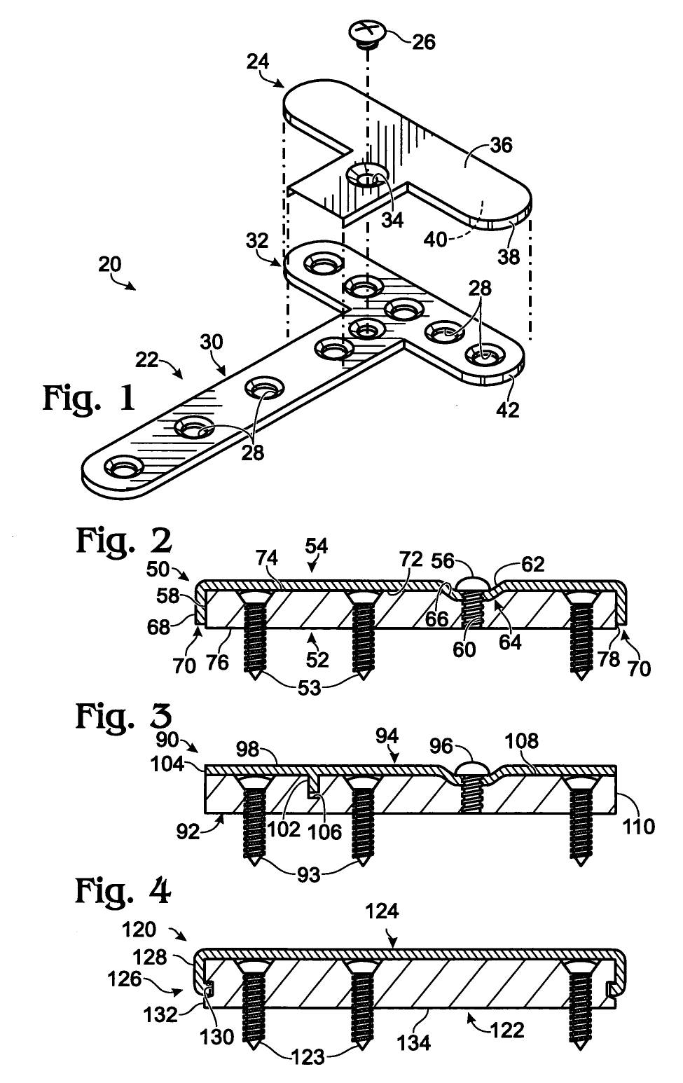

FIG. 1 shows an exploded view of an exemplary system 20 for fixing bones. System 20 may include a bone plate 22 and a cover or cover plate 24 that attaches to the bone plate via a fastener 26 (such as a screw). The cover plate is disposed adjacent and/or engages the outer surface of the bone plate and covers at least a portion of the bone plate's outer surface. The cover plate generally fits closely over the bone plate, generally following any nonplanar contour of the bone plate's outer surface, so that the increase in profile/thickness due to the cover plate is reduced or minimized. In some embodiments, the cover plate may have a relatively uniform thickness, such that the outer surface of the cover plate has a topography that substantially matches underlying outer surface regions of the bone plate.

Bone plate 22 may have any suitable structure to facilitate bone fixation. For example, the bone plate may have a plurality of openings 28 that receive fasteners to secure the bone plate to bone. One or more of the openings may be designed to receive cover fastener 26 that attaches the cover plate to the bone plate. The bone plate may include a shank portion 30 and a head portion 32 connected to the shank portion. In some embodiments, the shank portion may be attached to a shaft (diaphyseal) region of a bone, and the head portion may be attached to an end (metaphyseal) region of the bone.

Cover plate 24 may have any suitable structure to facilitate covering at least a portion of the bone plate. The cover plate may include, for example, at least one (or only one) opening 34 that receives a fastener (such as threaded fastener 26). Furthermore, the cover plate may include a body 36 and one or more flanges 38 projecting transversely from an inner surface 40 of the body. The flange(s) may extend over and/or engage an outer edge 42 (and/or an inner edge) of the bone plate to restrict pivotal movement of the bone plate and thus reduce or minimize the use of fasteners for mounting the cover plate onto the bone plate.

FIG. 2 shows another exemplary system 50 for fixing bone. The system may include a bone plate 52 secured to bone via bone screws 53. The system also may include a cover plate 54 secured over the bone plate using a threaded fastener 56, with the cover plate extending over an outer edge 58 (e.g., a side wall surface) of the bone plate.

Fastener 56 may be disposed in threaded engagement with an internal thread 60 formed in an opening of the bone plate, to lock the fastener to the bone plate, and/or the fastener may thread into bone to attach the cover plate to the bone plate via engagement with bone. Furthermore, fastener 56 may be disposed in threaded or nonthreaded engagement with the cover plate. In some examples, nonthreaded engagement may be preferable so that fastener 56 may act as a lag screw that compresses the cover plate against the bone plate as the lag screw is tightened. The cover plate may include a recessed region, such as a counterbore 62, that receives the head of the fastener, to reduce or eliminate the profile of the fastener above the cover plate. In addition, a counterbore region 64 of the cover plate may be received in an underlying, corresponding counterbore 66 of the bone plate.

The cover plate may extend any suitable distance over and along edge 58 of the bone plate. For example, the cover plate may include a flange 68 that creates a lip or rim 70 at the perimeter of the cover plate. The lip may extend over opposing regions of the edge of the bone plate, as shown here, or may extend over one of a pair of the opposing regions. The lip may extend from an inner surface 72 of the cover plate past an outer face 74 of the bone plate, to a position adjacent edge 58, and generally toward an inner face 76 of the bone plate. The lip may extend only partway toward the inner face, may extend to a position substantially flush with the inner face, or may extend beyond the inner face such that a distal region of the lip projects inward (toward bone) from the inner face of the bone plate. Furthermore, the lip may form a seam 78 with the bone plate such that the seam is disposed substantially at the edge of the bone plate and/or closer to the inner face than the outer face of the bone plate.

FIG. 3 shows another exemplary system 90 for fixing bone. The system may include a bone plate 92 secured to bone via bone screws 93. The system also may include a cover plate 94 secured over the bone plate using a threaded fastener 96, with the cover plate extending into the bone plate at a position spaced from the fastener. In particular, the cover plate may include a body 98 and at least one integral flange 102 extending from the body centrally of a perimeter 104 of the body. The flange(s) may be shaped to be received in an opening 106 (a through-hole or recess) of the bone plate, for example, a pin received in a correspondingly sized hole of the bone plate. Flange 102 may restrict pivotal motion of the cover plate in relation to the bone plate and may facilitate proper positioning of the cover plate over the bone plate. The cover plate may extend only along outer face 108 of the bone plate or also may bend inward near its perimeter, as in FIG. 2, to extend over an edge 110 of the bone plate. In other words, the cover plate may include a combination of perimeter and nonperimeter flanges.

FIG. 4 shows another exemplary system 120 for fixing bone. The system may include a bone plate 122 secured to bone via bone screws 123. The system also may include a cover plate 124 secured over the bone plate using a snap-fit mechanism 126. In particular, the cover plate may include a flange 128 that is received in a groove 130 formed in an edge 132 of the bone plate and/or the flange may extend over and around the edge to a position adjacent inner face 134 of the bone plate.

### II. BONE PLATES

Bone plates of the present teachings generally comprise any plate-like fixation device configured for attachment to bone. The bone plates generally have a low enough profile on bone that they can be used for internal fixation, that is, with the bone plates disposed mostly or completely under the skin of plate recipients. The bone plates may be of a sturdy yet malleable construction. Generally, the bone plates should be stiffer and stronger than the section of bone spanned by each plate, yet flexible (e.g., springy) enough not to strain the bone significantly. The bone plates may be configured to reduce irritation to the bone and surrounding tissue. For example, the bone plates may have a low and/or feathered profile to reduce their protrusion into overlying tissue and rounded, burr-free surfaces to reduce the effects of such protrusion.

The bone plates may be unitary or may include two or more discrete components. The two or more discrete components may be connected through a mechanical joint that enables translational and/or pivotal movement to adjust the shape and/or size of the bone plates. Further aspects of unitary and multi-component bone plates that are adjustable are described in the patent applications listed above under Cross-References to Related Applications, which are incorporated herein by reference, particularly U.S. patent application Ser. No. 10/716,719, filed Nov. 19, 2003; U.S. patent application Ser. No. 10/717,015, filed Nov. 19, 2003; U.S. patent application Ser. No. 717,399, filed Nov. 19, 2003; and U.S. patent application Ser. No. 10/717,402, filed Nov. 19, 2003.

A. Bone Plate Shape and Structure

The bone plates of the present teachings may have any shape suitable for use on their intended target bones. The bone plates may be shaped for use on any suitable bone or bones, including a bone of the arms (such as a humerus, a radius, an ulna, etc.), a bone of the legs (such as a femur, a tibia, a fibula, etc.), a bone of the hand (a carpal, metacarpal, or phalange), a bone of the foot (a tarsal, metatarsal, or phalange), a clavicle, a rib, a scapula, a pelvic bone, a vertebra, and/or the like. The bone may have any suitable condition to be treated, such as a fracture, a malunion, a nonunion, a cut (an osteotomy), a structural weakness, an undesirable length and/or angulation, and/or the like. The condition may affect any suitable portion of the bone, such as a diaphyseal (shaft) and/or a metaphyseal (end) region of the bone. In exemplary embodiments, the condition affects a distal portion of a radius bone.

The distal radius, as used herein, refers to any portion of the radius bone that is spaced from the proximal end of the radius bone. Generally, the distal radius refers to a distal portion that is less than about one-half or one-third the length of the radius bone. The bone plates of the present teachings may be configured preferably to fix radius bones having fractures or other discontinuities disposed in the distal about one-fourth of the radius, although they may be used more generally to repair any suitable fracture.

Each bone plate may be configured for use on any suitable side or sides of the body. For example, the bone plate may be configured for use on both the left side and right side of the body/skeleton, such as when the bone plate has bi-lateral mirror symmetry. Alternatively, each bone plate may be configured for use on either the left side or right side of the body/skeleton, but not both.

The bone plates may be shaped and sized for use on any position of a target bone. For example, a bone plate may be shaped for use near an end of a bone, with a shank or body portion of the bone plate disposed more centrally on a shaft of the bone, and a widened head portion of the bone plate disposed more towards a metaphyseal end of the bone. Alternatively, the bone plate may be shaped for use more centrally on only a shaft of the bone.

The bone plates may have any suitable contour. In some examples, the bone plates may be supplied in a pre-contoured configuration (e.g., by pre-operative bending and/or machining, among others) to include an inner surface that is complementary to an external, nonplanar surface region of a target bone, such as the distal radius (e.g., a distal volar, distal dorsal, distal lateral, and/or distal medial surface). The bone plates thus may be pre-contoured according to an average or representative surface geometry of a bone. Alternatively, or in addition, the bone plates may be contoured peri-operatively (e.g., by bending), to adjust their shape before and/or during their installation on bone, to improve, for example, the fit of the bone plates on a target bone for particular individuals.

The bone plates may include a proximal portion and a distal portion configured to have corresponding relative dispositions on the distal radius. The proximal portion thus may be configured to be disposed substantially proximal to a bone discontinuity, and the distal portion may be configured to be disposed substantially distal to a bone discontinuity, so that these portions are attached to the radius adjacent opposing sides of the discontinuity.

The proximal and distal portions may be connected to one another through a bridge or junction region of each bone plate. The junction region may be joined unitarily to each of the proximal and distal portions, to provide a plate member of unitary construction, or may provide a site at which discrete proximal and distal plate components are connected to each other, to provide a plate member of non-unitary construction. The junction region may be configured to allow proximal and distal portions of each plate to slide, bend, turn, and/or twist relative to one another. Alternatively, or in addition, the junction region may provide a site at which a guide device may be attached to the bone plate. Fasteners also or alternatively may be placed into bone from the junction region. However, in some cases, the junction region may span a discontinuity in bone so that fastener placement into bone from the junction region may be less desirable than in other portions of the plate.

The bone plates may be generally elongate (at least before bending), with a length L, a width W, and a thickness T. Here, length L≧width W>thickness T. In use, the long axis of the plates, and particularly of a proximal portion, may be aligned with the long axis of the radius bone and/or may extend obliquely and/or transversely relative to the long axis.

The thickness of the bone plates generally is defined by a distance between inner (bone-facing) and outer (bone-opposing) surfaces of the plates. The thickness may be generally constant, at least locally within the bone plate, such that the inner and outer surfaces of the bone plate are generally complementary. However, the thickness of the plates may vary according to the intended use, for example, to make the bone plates thinner as they extend over protrusions (such as processes, condyles, tuberosities, and/or the like), reducing their profile and/or rigidity, among others. The thickness of the bone plates also may be varied to facilitate use, for example, to make the plates thinner, to facilitate bending where they typically need to be contoured peri-operatively. In this way, the plates may be thicker and thus stronger in regions where they typically do not need to be contoured, for example, regions of the plates that are placed along the shaft of the bone, among others. In some examples, the proximal portion of each bone plate may be thicker than the distal portion and/or the bridge region disposed between the proximal and distal portions. A thinner bridge region may permit adjustment of the relative angular disposition of the proximal and distal portions by bending and/or twisting the plate at the bridge region. A thinner distal portion may reduce irritation by reducing the profile of this portion of the bone plate. In some examples, the proximal and distal portions may have about the same thickness, or the distal portion may be thicker than the proximal portion.

B. Bone Plate Apertures

The bone plates generally include a plurality of apertures (openings) configured to perform similar or different functions. The apertures may be adapted to receive fasteners for affixing the bone plates to bone. Alternatively, or in addition, one more of the apertures may be configured to receive a fastener and/or a flange portion of a cover plate to secure the cover plate to the bone plate and/or restrict its movement. The apertures also or alternatively may function to alter the local rigidity of the bone plate and/or to facilitate blood flow to a fracture or surgical site to promote healing, among others. In some examples, one or more apertures of a bone plate may be configured for coupling a guide device to the bone plate. The aperture(s) for the guide device also may be used for attachment of a cover plate after the guide device is removed. Each aperture of a bone plate may have any suitable shape, including non-elongate (such as circular) or elongate (such as oval, elliptical, rectangular, etc.). Apertures may be created and/or tapped (threaded) pre-operatively, such as during the manufacture of the plates, and/or peri-operatively, such as with the plates disposed on bone. Further aspects of tapping apertures peri-operatively are included in U.S. patent application Ser. No. 10/873,522, filed Jun. 21, 2004, which is incorporated herein by reference.

Individual apertures may be locking or nonlocking. Exemplary locking apertures include a thread, ridge, and/or lip for engaging complementary structure on a fastener, to restrict axial movement of the fastener into or out of the aperture. The thread and/or the wall of the aperture also may be configured to stop over-advancement of a fastener. For example, the thread may terminate in a dead end adjacent the inner surface of the plate, and/or the thread or aperture may taper inward toward the inner surface. Alternatively, or in addition, structure to stop over-advancement of the fastener may be included in the fastener. Other locking apertures are described in the patent applications listed above under Cross-References to Related Applications, which are incorporated herein by reference, particularly U.S. patent application Ser. No. 11/071,050, filed Feb. 28, 2005.

The bone plates may have one or more openings configured as slots. A slot is any opening having a length that is greater than its width. The slot may be linear, arcuate, or angled, among others. The slot may include a counterbore structure to receive a head of a bone screw. The counterbore structure may be configured, as in a compression slot, to exert a force generally parallel to the long axis of the slot when a bone screw is advanced against the counterbore structure. Slots may extend axially, that is, in general alignment with the long axis of the plate, or transversely, that is, substantially nonparallel to the long axis, that is, oblique to the long axis or orthogonal to the long axis. Each bone plate may have one or more axial slots and one or more transverse slots. The slots may be used to adjust the translational and/or angular disposition of each bone plate on bone. Further aspects of slots that may be included in the bone plates of the present teachings are described further in the patent applications listed above under Cross-References to Related Applications, which are incorporated herein by reference, particularly, U.S. patent application Ser. No. 10/717,015, filed Nov. 19, 2003.

The bone plates may be configured to receive wires. Each bone plate thus may include one or more holes (generally of smaller diameter) extending through the plate between inner and outer surfaces of the plate. Alternatively, or in addition, the bone plates may be configured to receive and retain wires that extend over (or under) the plates, rather than through the plates, from a region(s) of bone spaced from the plates. Further aspects of bone plates configured to secure wires are described in the patent applications listed above under Cross-References to Related Applications, which are incorporated herein by reference, particularly U.S. patent application Ser. No. 11/109,985, filed Apr. 19, 2005.

C. Plate Materials

A bone plate of the present teachings may be at least substantially formed of, or may include, any suitable biocompatible material(s) and/or bioresorbable (bioabsorbable) material(s). Exemplary biocompatible materials that may be suitable for the bone plate include (1) metals/metal alloys (for example, titanium or titanium alloys, cobalt-chrome alloys, stainless steel, etc.); (2) plastics (for example, ultra-high molecular weight polyethylene (UHMWPE), polymethylmethacrylate (PMMA), polytetrafluoroethylene (PTFE), polyetheretherketone (PEEK), and/or PMMA/polyhydroxyethylmethacrylate (PHEMA)); (3) ceramics (for example, alumina, beryllia, calcium phosphate, and/or zirconia, among others); (4) composites (for example, carbon-fiber composites); (5) bioresorbable materials or polymers (for example, polymers of α-hydroxy carboxylic acids (e.g., polylactic acid (such as PLLA, PDLLA, and/or PDLA), polyglycolic acid, lactide/glycolide copolymers, etc.), polydioxanones, polycaprolactones, polytrimethylene carbonate, polyethylene oxide, poly-β-hydroxybutyrate, poly-β-hydroxypropionate, poly-δ-valerolactone, poly(hydroxyalkanoate)s of the PHB-PHV class, other bioresorbable polyesters, and/or natural polymers (such as collagen or other polypeptides, polysaccharides (e.g., starch, cellulose, and/or chitosan), any copolymers thereof, etc.); and/or the like. In some examples, one or more of these materials may form the body of a bone plate and/or a coating thereon.

Further aspects of bone plates that may be suitable for use as bone plates of the present teachings are described in the patent applications listed above under Cross-References to Related Applications, which are incorporated herein by reference, particularly U.S. patent application Ser. No. 10/716,719, filed Nov. 19, 2003; U.S. patent application Ser. No. 10/717,015, filed Nov. 19, 2003; U.S. patent application Ser. No. 717,399, filed Nov. 19, 2003; U.S. patent application Ser. No. 10/717,402, filed Nov. 19, 2003; and U.S. patent application Ser. No. 10/731,173, filed Dec. 8, 2003.

### III. COVER PLATES

The fixation systems of the present teachings may include a cover plate that attaches to a bone plate. The cover plate may have any suitable shape, size, apertures, and/or flanges, and may be formed of any suitable material(s).

The shape and size of the cover plate may be selected according to the bone plate to which the cover plate is to be attached and the extent of the bone plate to be covered. The cover plate thus may be configured for use on only one side of a body (e.g., a left-sided cover plate for a left-sided bone plate) or may be configured for use on both the left and right side of the body (e.g., for attachment to a bilaterally symmetrical bone plate). The cover plate may have a footprint that corresponds in shape to a portion or all of the bone plate. The cover plate may extend over the outer surface of (i.e., may cover) any suitable portion or all of the bone plate. For example, the cover plate selectively may cover one end portion of the bone plate, such as selectively covering a head portion (or shank portion) of the bone plate. The cover plate may cover any suitable portion of the head portion, such as at least about one-half, substantially all, a majority of the apertures of the head portion, at least a distal row of apertures, and/or the like. The cover plate thus may extend at least to opposing edges of the bone plate (such as to opposing side wall surfaces of the head portion), to an end of the bone plate (such as a distal edge (e.g., an end wall surface) of the bone plate), and/or the like. In some embodiments, the cover plate may extend to and along a majority or at least substantially all (or all) of the perimeter of the head portion.

The cover plate may extend any suitable distance toward or over the edge of the bone plate. In some embodiments, the cover plate may be restricted to positions adjacent the outer face of the bone plate. Alternatively the cover plate may extend over an edge of the bone plate, by bending inward generally toward the bone plate and bone. Bending inward may be gradual, for example, a curved inward bend, and/or may be angular, such as a sharp inward bend. This inward bending may create a flange disposed at the perimeter of the cover plate that extends over and/or along the bone plate edge. A bone plate edge, as used herein, generally refers to side-wall regions and end-wall regions that extend around the perimeter of the bone plate, between the inner and outer faces of the bone plate. The bone plate edge thus may be arranged obliquely to the inner and outer faces of the bone plate and/or orthogonally thereto. The edge may include a sharp, angular transition between the inner and/or outer face, and/or a gradual, such as a radiused transition.

The cover plate may have any suitable thickness(es). For example, the thickness may be greater than, about the same as, or substantially less than the thickness of the corresponding bone plate. In some embodiments, the thickness may be less than about one-half or less than about one-fourth the thickness of the bone plate, to reduce or minimize the increase in overall thickness introduced by the cover plate. The thickness of the cover plate may be substantially the same (uniform) throughout the cover plate or may vary, for example, such that the cover plate is thicker or thinner toward the perimeter of the cover plate. Alternatively, the thickness may vary so that the outer surface of the cover plate is less angular than the inner surface, to smooth out the surface contours of the outer surface. Generally, the inner surface of the cover plate is substantially complementary to the outer surface of the bone plate, such that the cover plate fits closely onto and engages the bone plate, to reduce or minimize unnecessary increases in overall thickness created by a poor fit and/or to reduce cover plate slippage relative to the bone plate. The thickness of the cover plate may decrease (e.g., by tapering or feathering) toward its perimeter, or portions thereof, particularly if the cover terminates part way across the outer surface of the bone plate, to reduce any discontinuity in the thickness of the bone plate plus cover plate versus the bone plate alone.

The cover plate may include any suitable number of apertures (openings). Each aperture may be circular or noncircular (e.g., oval) and may be locking (e.g., threaded) or nonlocking. Each aperture may include or lack a counterbore disposed toward an outer (or inner) surface of the cover plate. The apertures may be sized to receive a screw, a wire, a pin, and/or the like.

The cover plate may include any suitable number of flanges. The flange(s) may be integral to the cover plate, such that the cover plate is of unitary structure. The flange thus may be formed from the same piece of material as the body of the cover plate, such as by bending, molding, or machining the piece of material, or may be formed from a separate piece of material that is attached permanently to the cover plate's body (e.g., by welding or with an adhesive, among others).

Each flange may extend from any suitable position on the body of the cover plate, generally from the perimeter of the body (to form a perimeter flange) and/or central to the perimeter (to form an internal flange). A perimeter flange may extend along any portion of the perimeter of the cover plate's body. For example, along opposing sides of the body, along an end of the body, and/or the like. The perimeter flange may be continuous or may be a plurality of distinct flanges extending at discrete positions from the body. An internal flange may have any suitable shape and size. For example, the internal flange may be sized and positioned according to an opening(s) of a corresponding bone plate and thus may extend into and/or substantially through the opening.

A cover plate may be formed of, or may include, any suitable material. Exemplary materials that may be suitable are any of the materials described above in Section II for bone plates. The cover plate may be formed of the same material as a corresponding bone plate or may be formed of a different material. For example, the bone plate may be formed of metal and the cover plate may be formed of plastic (or vice versa), or the bone plate may be formed of a nonbioresorbable material and the cover plate may be formed of a bioresorbable material (or vice versa). In some examples, the bone plate and its corresponding cover plate may be formed of different metals (including different metal alloys). For example, the bone plate may be formed of titanium or a titanium alloy and the cover plate may be formed of stainless steel or a cobalt-chrome alloy. Construction with different metals may allow the bone plate and cover plate to benefit from the distinct properties of the metals. In particular, titanium and/or a titanium alloy may offer a suitable strength and/or flexibility for the bone plate and another metal (such as those listed above) may provide a less abrasive surface for the cover plate than a titanium/titanium alloy of the bone plate, to reduce tendon irritation produced by contact with the bone plate.

### IV. KITS FOR BONE FIXATION

The systems of the present teachings may provide kits for fixing bones. The kits may include one or more bone plates, one or more cover plates that fit onto and attach to the bone plates, fasteners (such as bone screws, wires, or the like) for securing the bone plate(s) and cover plate(s) to bone and/or each other, a guide device, a drill(s), one or more clamps, instructions for use, and/or the like. Some or all of the components of each kit may be provided in a sterile condition, such as packaged in a sterile container.

In some examples, the kits may include a set of two or more cover plates. The cover plates may differ in size (such as the proportion of a corresponding bone plate covered), handedness (such as cover plates for use on left and right bone plates), site of plate attachment, thickness, and/or the like.

### V. APPLICATION OF FIXATION SYSTEMS

The fixations systems of the present teachings may provide methods of fixing bones. The methods may include any combination of the following steps, performed in any suitable order, and any suitable number of times, including once or more than once: (1) select a bone to be fixed, (2) select a bone plate for the bone, (3) select a cover plate for the bone plate, (4) secure the bone plate to the bone, and (5) cover at least a portion of the bone plate with the cover plate. Further aspects of the steps are described below.

A bone to be fixed may be selected. The bone may have any suitable discontinuity, including a fracture, a cut (e.g., produced by an osteotomy), a malunion, a nonunion, etc. The fracture may be a single break or a plurality of connected or separate breaks. The bone may be a long bone or another bone of the skeleton. In some examples, the bone is a radius bone, particularly a radius bone that has sustained a fracture, or two or more fractures to a distal region of the bone. The bone discontinuity may be reduced, e.g., the fracture set. Reduction may be performed before and/or after the bone plate and/or cover plate are applied to the bone. Selecting a bone to be stabilized also may include creating an incision through soft tissue on the volar, dorsal, lateral, and/or medial side of the bone, to access the bone. This and other suitable steps of the methods may be performed under sterile conditions and/or in a sterile field, for example, during surgery in an operating room.

A bone plate for the bone may be selected. The bone plate may be selected according to the bone to be stabilized and thus may have a size and shape corresponding to the bone. For example, the bone plate may be contoured so that its inner surface is nonplanar and is complementary to a nonplanar, exterior surface region of the bone. In some examples, the bone plate may be pre-contoured (e.g., by bending, machining, and/or casting, among others) according to an average anatomy of a bone within a population. The bone plate may be configured for use on both sides of the skeleton, or may be configured for use on a right bone or a left bone, but not both. In some examples, the bone plate may include indicia (e.g., one or more alphanumeric characters, one or more words, a color, a bar code, etc.) to identify the bone plate, the bone for which the bone plate is configured, the size of the bone plate, the handedness of the bone plate, and/or the like.

A cover plate for the bone plate may be selected. The cover plate may be selected from a set of two or more cover plates of different sizes, shapes, contours, handedness, etc. Accordingly, the cover plate may include indicia, as described above for bone plates, to facilitate selection of a suitable cover plate. The cover plate may be shaped to fit onto the bone plate and thus may be selected according to its ability to fit onto the bone plate. The cover plate may be shaped peri-operatively (for example, bent before and/or during a surgery in which the bone plate is installed) and/or may be pre-shaped, e.g., during manufacture. In some embodiments, the cover plate may be pre-shaped but also must be shaped peri-operatively according to a change in shape of the bone plate introduced peri-operatively. The cover plate thus may be formed to be malleable.

The bone plate may be disposed on the bone and secured. The bone plate may be disposed on any suitable surface along and/or around the bone, such as on a distal volar or distal dorsal surface of a radius bone, among others. The bone plate may be disposed such that the bone plate spans a fracture in the bone, such as an extra-articular (and/or intra-articular) fracture. The bone plate may be secured by placement of fasteners through openings of the bone plate. The fasteners may include wires and/or bone screws, among others. The fasteners may be placed through bone plate openings disposed on opposing sides (or only one side) of a discontinuity in the bone. Placement of fasteners may be facilitated with a guide device that directs placement of a wire, a drill, and/or a bone screw, among others.

At least a portion of the bone plate may be covered with the cover plate. The bone plate may be covered before or after the bone plate is disposed on and/or secured to bone, but generally after the bone plate is secured if bone plate openings and their associated bone screws are being covered. The cover plate may be attached to the bone plate by any suitable coupling mechanism, such as placement of one or more threaded fasteners into threaded engagement with the cover plate, bone plate, and/or bone, among others. In some examples, the cover plate may be coupled to the bone plate after a guide device has been removed from the bone plate, and one or more of the same bone plate openings may be used to secure the guide device and the cover plate.

The bone plate and/or cover plate may be removed at any suitable time. In some examples, the bone plate and cover plate may be left in place indefinitely. In some examples, the cover plate may be removed selectively and the bone plate left in position for a longer period of time. In some examples, the bone plate and cover plate may be removed at a suitable time, such as after sufficient healing has occurred.

### VI. EXAMPLE

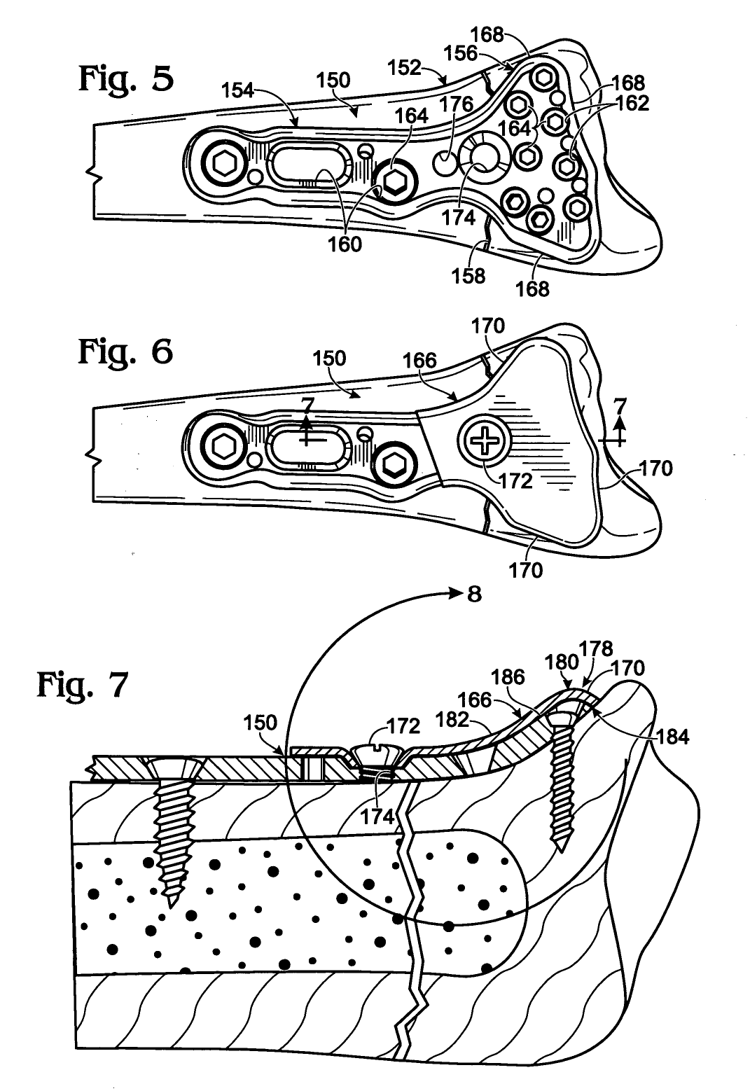

The following example describes selected aspects of an exemplary system for fixation of bones, particularly a distal portion of a radius bone, using a bone plate and a cover plate that mounts onto the bone plate; see FIGS. 5-9. This example is included for illustration and is not intended to limit or define the entire scope of the present teachings.

FIG. 5 shows an exemplary bone plate 150 secured to a distal region of a fractured radius bone 152, on the volar side of the radius bone. Bone plate 150 may include a shank portion 154 secured more proximally on the radius bone and a head portion 156 connected to the shank portion and secured more distally to the radius bone, such that the plate spans one or more fractures 158. The shank and head portions each may include a plurality of openings (160 and 162, respectively) through which bone screws 164 may be placed into the radius bone. Each bone screw may lock to the bone plate or more be installed in nonlocking engagement with the bone plate. Further aspects of the structure and use of bone plate 150 are described in the following patent applications, which are incorporated herein by reference: U.S. patent application Ser. No. 10/716,719, filed Nov. 19, 2003; U.S. patent application Ser. No. 10/968,850, filed Oct. 18, 2004; and U.S. patent application Ser. No. 11/109,985, filed Apr. 19, 2005.

Head portion 156 may have a nonplanar inner surface that matches that of the volar (or dorsal) surface of the distal radius, and a nonplanar outer surface corresponding to the inner surface. The nonplanar outer surface and/or its position near the end of the bone may cause the head portion of the bone plate to irritate tendons extending over the head portion, particularly as the adjacent wrist joint is operated.

FIG. 6 shows bone plate 150 with an exemplary cover plate 166 secured to the bone plate. Cover plate 166 may extend to and along all or a part of perimeter 168 of the head portion (see FIGS. 5 and 6). The cover plate thus may have a footprint that corresponds closely to the head portion of the bone plate. However, the cover plate may extend somewhat into the shank portion of the bone plate or may extend incompletely to the proximal end of the head portion of the bone plate, among others. The footprint of the cover plate may be smaller than, the same size as, or enlarged relative to a corresponding portion of the footprint of the bone plate. In the present illustration, the footprint is enlarged according to the local thickness of a perimeter flange 170 of the cover plate (see FIG. 9 also).

The cover plate may be secured to the bone plate with a threaded fastener 172 received in a threaded opening 174 of the bone plate (see FIGS. 5-7). In some embodiments, the cover plate also may include an internal flange(s) (e.g., a pin or boss) that fits into an aperture (such as aperture 176 of FIG. 5) disposed in the head portion and/or shank portion of the bone plate (and/or in a bridge region generally between the head and shank portions). The use of an internal flange and/or perimeter flange on the cover plate may allow the cover plate to be secured to the bone plate with only one fastener, because the flange may restrict pivotal motion through engagement with the bone plate.

FIG. 7 shows a sectional view of the bone plate, cover plate, and radius bone, taken generally along line 7-7 of FIG. 6. The cover plate may extend over an edge of the cover plate, such as a distal edge region 178 disposed at the distal end of the bone plate. Accordingly, the cover plate may bend inward along an arcuate path, shown at 180, to create a radiused shaped as the cover plate transitions from a body 182 of the cover plate to its connected perimeter flange 170. By extending over the edge of the bone plate, a seam 184 formed at the surface between the bone plate and cover plate may be disposed in a spaced relation from an outer face 186 of the bone plate, to reduce or minimize the chance of additional tendon abrasion produced by tendon contact with the seam.

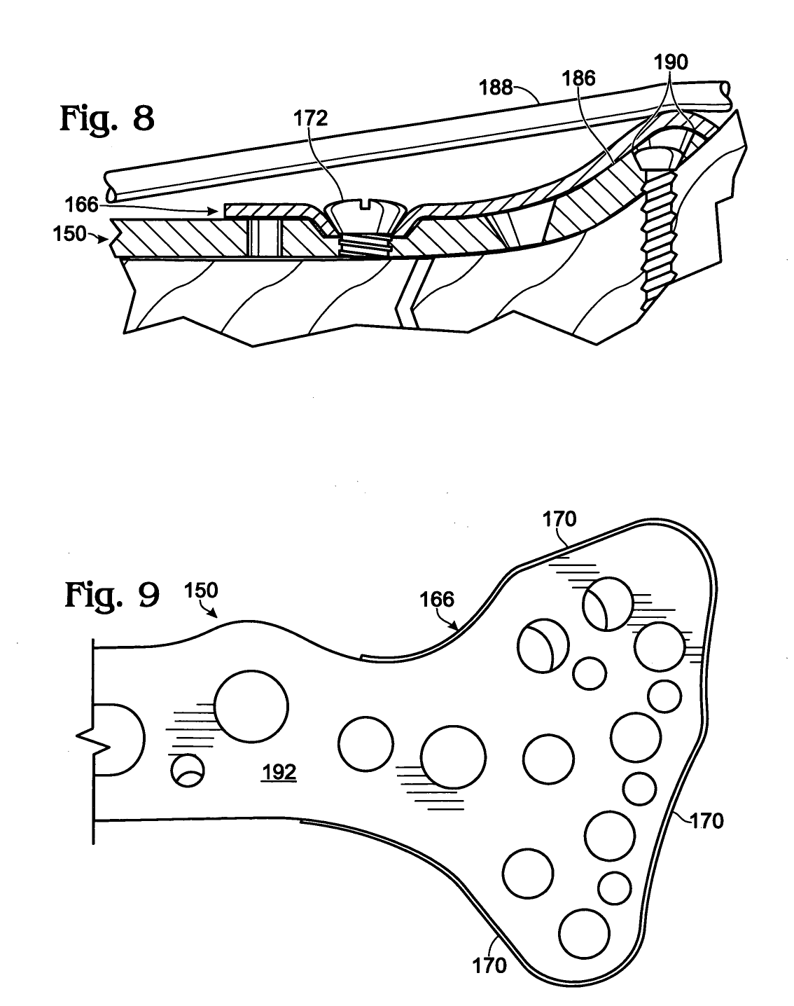

FIG. 8 shows selected portions of FIG. 7, taken generally at “8” in FIG. 7, with a tendon 188 extending over bone plate 150 and cover plate 166. Juxtaposition of the tendon with the bone plate may be facilitated by pre-operative or peri-operative injury to muscle, such as injury to the pronator quadratus muscle on the volar side of the distal radius. In the absence of the cover plate, tendon 188 may rub against outer face 186 of the bone plate, particularly near the distal end of the bone plate. The degree and/or severity of rubbing may be related to the distal slope of the volar radial surface, which varies substantially within the population. (A flatter volar surface may correlate with increased tendon irritation.) Tendon irritation also may be a problem if the bone plate is installed on the dorsal side of the radius, where four to five tendons may run over the outer surface of the bone plate. In any case, the tendon may be irritated by any abrasiveness of the material forming the outer face and/or by edges 190 introduced by the apertures, particularly apertures disposed near the distal end of the bone plate. However, cover plate 166 may reduce the amount of tendon abrasion by reducing the number of aperture edges to which the tendon is exposed and/or by providing a less abrasive surface material for contact with the tendon.

FIG. 9 shows bone plate 150 and cover plate 166 viewed toward an inner face 192 of the bone plate in the absence of bone and fasteners. Perimeter flange 170 may extend along the entire perimeter of the head portion (or any suitable portion thereof). Furthermore, the perimeter flange (and/or the cover plate) may be thin enough that the increase in overall width and thickness of the fixation device (bone plate and cover plate) produced by installation of the cover plate is minimal.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

I claim:

1. A device for fixing bone, comprising:
a bone plate defining a plurality of openings for receiving fasteners that attach the bone plate to bone, the bone plate including an edge extending along a perimeter of the bone plate; and
a cover plate that fits onto and attaches to the bone plate, the cover plate including a body having an inner surface and also including at least one integral flange extending transversely from the inner surface such that the flange extends into the bone plate and/or over the edge of the bone plate when the cover plate fits onto the bone plate, to restrict pivotal movement of the cover plate by engagement of the flange with the bone plate.

2. The device of claim 1, wherein the at least one integral flange includes a pin received in an opening of the bone plate when the cover plate fits onto the bone plate.

3. The device of claim 1, wherein the body has a perimeter, and wherein the at least one integral flange extends from the inner surface at the perimeter.

4. The device of claim 1, wherein the bone plate includes a head portion having an outer surface, and wherein the cover plate mostly or completely covers the outer surface of the head portion.

5. The device of claim 4, wherein the bone plate includes a shank portion connected to the head portion, and wherein the cover plate selectively covers the head portion relative to the shank portion.

6. The device of claim 4, wherein the head portion includes a subset of the plurality of openings, and wherein the cover plate covers more than one-half of the openings of the subset.

7. The device of claim 1, wherein the bone plate is shaped to fit onto a distal region of a radius bone.

8. The device of claim 1, wherein the cover plate is substantially less than one-half as thick as the bone plate.

9. The device of claim 1, wherein the bone plate and the cover plate are formed of different materials.

10. The device of claim 1, wherein the bone plate is formed of titanium or a titanium alloy, and wherein the cover plate is formed of stainless steel or a cobalt-chrome alloy.

11. The device of claim 1, wherein the bone plate has a nonplanar outer surface, and wherein the cover plate has an outer surface with a topography that substantially matches a region of the nonplanar outer surface.

12. A device for fixing bone, comprising:
a bone plate defining a plurality of openings for receiving fasteners that attach the bone plate to bone, the bone plate including opposing inner and outer faces; and
a cover plate that fits onto and attaches to the bone plate to create a seam disposed closer to the inner face than the outer face.

13. The device of claim 12, wherein the bone plate includes a head portion having a perimeter, and wherein the seam extends along a majority of the perimeter.

14. The device of claim 12, wherein the bone plate is shaped to fit onto a nonplanar, volar or dorsal surface region of a distal radius bone.

15. The device of claim 12, wherein the bone plate has a distal edge, wherein the distal edge has a length and wherein the cover plate extends over a substantial portion of the length of the distal edge.

16. A method of fixing a bone, comprising:
attaching a bone plate with a head portion to a bone; and
covering a majority of the head portion of the bone plate with a cover plate secured to the bone plate such that the cover plate extends over an edge of the bone plate.

17. The method of claim 16, wherein the step of attaching a bone plate includes a step of attaching a bone plate to a distal region of a radius bone.

18. The method of claim 16, the bone plate including a head portion having an outer surface, wherein the step of covering includes a step of at least mostly covering the outer surface of the head portion with the cover plate.

19. The method of claim 16, wherein the step of covering includes a step of covering the head portion with a cover plate having an outer surface with a topography that corresponds to an outer surface region of the bone plate.

20. The method of claim 16, wherein the step of covering produces a substantially uniform increase in thickness of most or all of the head portion.
